(19) United States
(12) Patent Application Publication
(10) Pub. No.: US 2004/0044371 A1
(43) Pub. Date: Mar. 4, 2004

(54) Defibrillator with H-bridge output circuit referenced to common ground

(75) Inventors: Paul S. Tamura, Seattle, WA (US); D. Craig Edwards, Fall City, WA (US); Joseph L. Sullivan, Kirkland, WA (US)

Correspondence Address: INGRASSIA FISHER & LORENZ, P.C., 7150 E. CAMELBACK, STE. 325, SCOTTSDALE, AZ, 85251, US

(73) Assignee: Medtronic Physio-Control Manufacturing Corp.

(21) Appl. No.: 10/236,057

(22) Filed: Sep. 4, 2002

(51) Int. Cl. A61N 1/39

(52) U.S. Cl. 607/5

(57) ABSTRACT

An external defibrillator having an output circuit that allows a defibrillation pulse to be discharged to a patient is provided. The output circuit, charging circuit, preamplifier circuit, impedance measurement circuit, energy storage device, battery, and measurement and control circuits of the defibrillator are all referenced to a common ground. The use of a common ground is simpler and less expensive than previous designs which utilized isolation stages and circuits for isolating the high and low voltage circuitry. The output circuit is in the form of an H-bridge which contains three SCR legs and one IGBT leg. Each of the legs contains a single semiconductor switch. The IGBT is placed in the northwest leg of the H-bridge. The two lower legs each contain SCRs, one or both of which may be driven by DC gate drive signals.

### FIELD OF THE INVENTION

This invention relates generally to apparatus for generating defibrillation waveforms, and more particularly to a circuit for generating a defibrillation waveform in an external defibrillator.

### BACKGROUND OF THE INVENTION

One of the most common and life-threatening medical conditions is ventricular fibrillation, a condition where the human heart is unable to pump the volume of blood required by the human body. The generally accepted technique for restoring a normal rhythm to a heart experiencing ventricular fibrillation is to apply a strong electric pulse to the heart using an external cardiac defibrillator. External cardiac defibrillators have been successfully used for many years in hospitals by doctors and nurses, and in the field by emergency treatment personnel, e.g., paramedics.

Conventional external cardiac defibrillators first accumulate a high-energy electric charge on an energy storage capacitor. When a switching mechanism is closed, the stored energy is transferred to a patient in the form of a large current pulse. The current pulse is applied to the patient via a pair of electrodes positioned on the patient's chest. The switching mechanism used in most contemporary external defibrillators is a high-energy transfer relay. A discharge control signal causes the relay to complete an electrical circuit between the storage capacitor and a wave shaping circuit whose output is connected to the electrodes attached to the patient.

certain studies indicate that there may be advantages to applying a biphasic rather than a monophasic waveform to the patient. For example, certain research indicates that a biphasic waveform may limit the resulting heart trauma associated with the defibrillation pulse. An H-bridge output circuit may be used for applying a biphasic defibrillation pulse.

The American Heart Association has recommended a range of energy levels for the first three defibrillation pulses applied by an external defibrillator. The recommended energy levels are: 200 joules for a first defibrillation pulse; 200 or 300 joules for a second defibrillation pulse; and 360 joules for a third defibrillation pulse, all within a recommended variance range of no more than plus or minus 15 percent according to standards promulgated by the Association for the Advancement of Medical Instrumentation (AAMI). These high-energy defibrillation pulses are required to ensure that a sufficient amount of the defibrillation pulse energy reaches the heart of the patient and is not dissipated in the chest wall of the patient.

Some implantable defibrillators, such as those shown in U.S. Pat. Nos. 5,083,562 and 4,880,357, use a bridge circuit with multiple silicon-controlled rectifiers (SCRs) to generate a biphasic waveform. Because implantable defibrillators only apply a low energy defibrillation pulse having a maximum energy of approximately 35 joules, however, the output circuit in implantable defibrillators is not adaptable for use in the external defibrillator. A 200-joule energy pulse applied to an implantable defibrillator bridge circuit may overload the bridge circuit components and cause the circuit to fail.

In addition, conventional external defibrillator circuits have typically been complex and expensive, with separate isolated circuitry required for the low voltage control circuitry and the high voltage defibrillation circuitry, due in part to the components required to conduct the large energy pulses that are generated in external defibrillators. It would be desirable to reduce the complexity and expense of such external defibrillator circuits, and to improve their efficiency.

The present invention is directed to providing apparatus that overcome the foregoing and other disadvantages. More specifically, the present invention is directed to an output circuit for an external defibrillator that is capable of applying a high-energy biaphasic defibrillation pulse to a patient, and which has reduced complexity and improved efficiency over prior external defibrillators.

### SUMMARY OF THE INVENTION

An external defibrillator having an output circuit that allows a defibrillation pulse to be discharged to a patient from an energy storage device, preferably an energy storage capacitor, is disclosed. In accordance with one aspect of the invention, the output circuit is referenced to a common ground in the defibrillator. In the defibrillator, the preamplifier, impedance measurement circuit, charging circuit, battery, energy storage device and measurement and control circuits are all referenced to a common ground without requiring the commonly used isolation stages and circuits. For example, certain prior art defibrillators have utilized isolation circuits for circuits such as the preamplifier or output circuits. It will be appreciated that the utilization of a common ground for the high and low voltage circuitry is advantageous in that the resulting circuit design is simpler and less expensive than prior art designs.

In accordance with another aspect of the invention, the output circuit includes four legs arrayed in the form of an “H” (hereinafter the “H-bridge output circuit”). Each leg of the output circuit contains a solid-state switch. By selectively switching on pairs of switches in the H-bridge output circuit, a biphasic defibrillation pulse may be applied to the patient.

In accordance with another aspect of the invention, the switches in three of the legs of the H-bridge output circuit are silicon controlled rectifiers (SCRs). Preferably, only a single SCR is used in each leg. The switch in a fourth leg is an insulated gate bipolar transistor (IGBT). In one embodiment, only a single IGBT is used in the fourth leg. The use of single SCR and IGBT switches in each leg simplifies the circuit as compared to the use of semiconductor modules that are large and expensive or as compared to the use of lower voltage parts, which must be stacked. The use of three SCR legs further reduces the size, weight, and cost of the H-bridge output circuit in comparison with an implementation using two SCR and two IGBT legs. The use of a single IGBT in a leg of the H-bridge (as opposed to two or more IGBTs in series) also greatly simplifies the drive circuitry required to turn on and off the IGBT.

In accordance with another aspect of the invention, two of the SCR legs of the H-bridge output circuit are the two lower H-bridge legs, and a DC gate drive signal may be utilized to drive one or both of the SCR switches. Prior art defibrillators have typically isolated the H-bridge from the control circuit ground potential. This configuration has required a transformer to couple a drive signal to the SCR gates, and because the transformers are unable to pass the DC signals, the gate has been driven with AC signals. The utilization in the present invention of a common ground for both the high-voltage and low-voltage circuitry allows the gates of one or both of the SCR switches to be driven directly from field effect transistors (FETs) with a DC signal.

In accordance with another aspect of the invention, the IGBT leg is made to be the northwest leg of the H-bridge. Certain prior art defibrillators have placed the IGBT in the southeast leg. Making the northwest leg the IGBT leg helps avoid a design issue that occurs when attempting to modify certain prior art defibrillator configurations to meet the present design requirements. More specifically, in the present configuration, a current path can exist from the midpoint of the H-bridge through the preamp protection resistors (in one embodiment 12 kohms) to ground. The amount of current flowing through this path (in one embodiment 170 mA) is negligible compared with the current delivered to the patient (in one embodiment greater than 10 amps), but is sufficient to create a complication in the operation of the SCRs. More specifically, utilizing a prior art configuration where the IGBT is in the southeast leg, a current through this path (as noted above in one embodiment 170 mA), would flow through the SCRs at the top of the H-bridge. Once one of these SCRs was turned on, it could not be turned off again until the capacitor was mostly discharged because there is no mechanism for shutting off the current through the preamp path. The utilization of the IGBT in the northwest leg of the H-bridge allows the current through the preamp path to be shut off (along with the current through the patient) at the end of the first phase of the defibrillation pulse.

In accordance with still another aspect of the invention, a single power switch is utilized in each of the legs of the H-bridge output circuit, and is included in a single integrated surface mountable module. The use of single semiconductor switches in a single package simplifies the assembly and manufacturing of the defibrillator device.

In accordance with another aspect of the invention, the H-bridge output circuit is capable of conducting a biphasic waveform of 200 or more joules from the energy storage capacitor to the patient. Preferably, the H-bridge output circuit is capable of conducting a biphasic waveform equal to 360 joules, the industry standard for monophasic waveforms and the recommended level for a third defibrillation pulse by the American Heart Association. To store sufficient energy for such a biphasic defibrillation pulse, the size of the energy storage capacitor may in one embodiment fall within a range from 150 uF to 200 uF.

Moreover, in addition to being able to conduct a high energy defibrillation pulse of 200 to 360 joules, the H-bridge output circuit is also capable of conducting a low energy defibrillation pulse. In one embodiment, a lower energy defibrillation pulse of 150 joules may be delivered, while in other embodiments the defibrillator of the invention could also be used at a general lower energy range such as 1 to 50 joules. Some types of low energy defibrillation pulses are required when, for example, internal paddles are coupled to the defibrillator for use in surgery to directly defibrillate the heart, or for pediatric defibrillation, or for cardioversion of some arrhythmias in both pediatrics and adults.

In accordance with another aspect of the invention, a gate drive circuit biases on the IGBT in the first leg with a sufficient voltage over a short interval to allow the leg to conduct a high current without being damaged. In one embodiment, the leg can conduct a current of at least approximately 200 amps. Biasing the IGBT in this manner allows the IGBT to withstand a high-energy discharge such as occurs when a low resistance load is placed at the output of the circuit.

In accordance with still another aspect of the invention, all of the output circuit switches are selected to have sufficient current conducting capability to allow the switches in two of the legs on the same side of the H-bridge to provide a shorted path for the discharge of unwanted energy from the energy storage capacitor. The use of two legs on one side of the H-bridge to discharge the capacitor eliminates the need for an additional discharge circuit to perform this internal energy dump function. In addition, the H-bridge circuit is able to perform the internal energy dump quickly and accurately using advantageous component values that would not be practical to implement in a separate discharge circuit. For example, the H-bridge circuit is able to perform an internal dump in less than one second through the use of a resistive component with a value of less than 100 ohms. Also, because the H-bridge circuit is used for both the internal dump and defibrillation pulse operations, the resistive component of the H-bridge circuit serves to both absorb energy during the internal dump and also to limit current during the defibrillation pulse. The resistive value is selected to be small enough to allow sufficient current to provide both an effective defibrillation pulse and a fast internal energy dump, while also being large enough to limit the current so as to protect the switches of the H-bridge circuit. The resistive component is also selected to have a high thermal capacity so that it can withstand the heat produced by the high currents that result during the H-bridge internal dump and defibrillation pulse operations.

It will be appreciated that the disclosed defibrillator with a unique H-bridge output circuit that is referenced to a common ground with the preamplifier and charging circuits is advantageous in that it is simpler, less expensive, and operates more effectively than prior art defibrillators.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a block diagram of an external defibrillator where the output circuit, charging circuit, and preamp circuit are referenced to a common ground in accordance with the present invention;

FIG. 2 is a more detailed block diagram of the external defibrillator of FIG. 1;

FIG. 3 is a schematic diagram of a preferred embodiment of the output circuit and transfer relay of the defibrillator of FIG. 2;

FIG. 4 is a block diagram of the charging circuit of the defibrillator of FIG. 2;

FIG. 5 is a schematic diagram of a preferred embodiment of the charging circuit of FIG. 4;

FIG. 6 is a block diagram of the preamp ECG and impedance drive and measurement circuit of FIG. 2; and

FIGS. 7A-7C are schematic diagrams of a preferred embodiment of the preamp ECG and impedance drive and measurement circuitry of FIG. 6.

### DETAILED DESCRIPTION OF THE PREFERRED EMBODIMENT

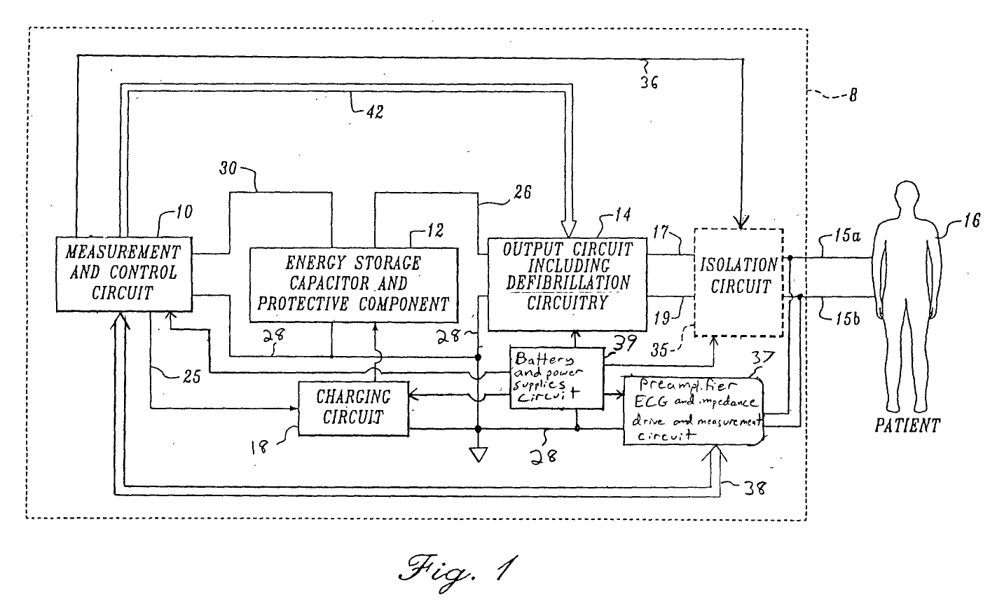

FIG. 1 is a block diagram of an external defibrillator 8 that is connected to a patient 16. The defibrillator includes a measurement and control circuit 10 that is connected to an energy storage capacitor and protective component 12 via a charging circuit 18. During the operation of the defibrillator, the measurement and control circuit 10 controls the charging circuit 18 via a control line 25 to charge the energy storage capacitor to a desired voltage level. Feedback on the voltage level of the energy storage capacitor is provided to the measurement and control circuit 10 on a pair of lines 28 and 30.

After charging to a desired level, the energy stored in the energy storage capacitor may be delivered to the patient 16 in the form of a defibrillation pulse. The energy storage capacitor and protective component 12 is connected by lines 26 and 28 to an output circuit 14. Output circuit 14 includes defibrillation circuitry. The measurement and control circuit 10 is connected to the output circuit 14 by a control bus 42 and to an isolation circuit 35 by a control line 36. Application of appropriate control signals over the control bus 42 and control line 36 causes the output circuit 14 to conduct energy from the energy storage capacitor. The energy is delivered to the patient 16 attached to the defibrillator 8 over a set of patient apex and sternum lines (aka electrodes) 15a and 15b. The apex line 15a is attached to an apex line 17 in output circuit 14 through the isolation circuit 35. The sternum line 15b is attached to a sternum line 19 in output circuit 14 through the isolation circuit 35.

The measurement and control circuit 10 also controls and receives measurements through a bus line 38 from a preamplifier ECG and impedance drive and measurement circuit 37. The preamplifier ECG and impedance drive and measurement circuit 37 is coupled to the apex and sternum lines 15a and 15b. The preamplifier ECG and impedance drive and measurement circuit 37 provides measurements of the ECG and impedance of the patient 16.

A battery and power supplies circuit 39 includes a battery or similar power source (e.g., a charge pack) for powering the defibrillator 8. The battery and power supplies circuit 39 provides power to the other circuit components, including the measurement and control circuit 10, the output circuit 14, the charging circuit 18, the isolation circuit 35, and the preamplifier ECG and impedance drive and measurement circuit 37. The battery and power supplies circuit 39 is referenced to the common ground 28.

As illustrated in FIG. 1, the measurement and control circuit 10, energy storage capacitor 12, output circuit 14, charging circuit 18, preamplifier ECG and impedance drive and measurement circuit 37, and the battery and power supplies circuit 39 are all referenced to the common ground 28. This is in contrast to certain prior art defibrillators which utilize isolation circuits for various circuits such as the output circuit or preamplifier. It will be appreciated that the utilization of a common ground rather than requiring isolation circuitry between the high and low voltage circuitry is advantageous in that it results in simpler and less expensive circuitry.

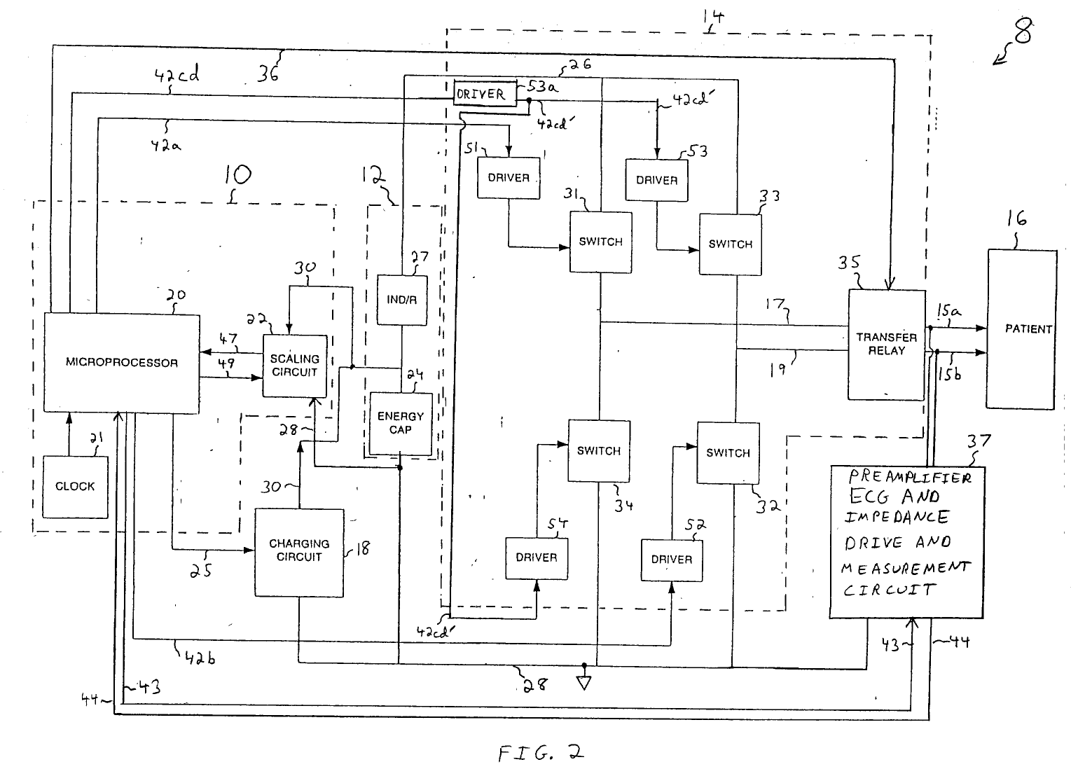

FIG. 2 is a more detailed block diagram of the external defibrillator 8 of FIG. 1, according to one embodiment of the present invention. The defibrillator 8 is connected to a patient 16 and includes a microprocessor 20 that is connected to an energy storage capacitor 24 via a charging circuit 18. It will be appreciated by those skilled in the art that energy storage capacitor 24 may be implemented with a multi-capacitor network (i.e., with capacitors connected in series and/or parallel). During the operation of the defibrillator 8, microprocessor 20 controls charging circuit 18 using a signal on a control line 25 to charge energy storage capacitor 24 to a desired voltage level. To monitor the charging process, microprocessor 20 is connected to a scaling circuit 22 by a measurement line 47, and by a control line 49. It will be understood that while single measurement and control lines are shown, multiple lines may be used. Scaling circuit 22 is connected to energy storage capacitor 24 by a bridge line 28, which connects to the negative lead of energy storage capacitor 24, and by a line 30, which connects to the positive lead of the capacitor. As will be described in more detail below, the bridge line 28 serves as a common ground for the defibrillator 8. A clock 21 is also connected to microprocessor 20.

After charging to a desired level, the energy stored in energy storage capacitor 24 may be delivered to patient 16 in the form of a defibrillation pulse. The H-bridge output circuit 14 is provided to allow the controlled transfer of energy from energy storage capacitor 24 to patient 16. H-bridge 14 is an output circuit that includes four switches 31, 32, 33, and 34, which are driven by four driver circuits 51, 52, 53, and 54, respectively. Driver circuits 53 and 54 are further driven by a driver circuit 53a, as will be described in more detail below. Each of the switches 31, 32, 33 and 34 is connected in a leg of the output circuit that is arrayed in the form of an “H”. Switches 31 and 33 are coupled through a protective component 27 to the positive lead of the energy storage capacitor 24 by a bridge line 26. Protective component 27 limits the current and voltage changes from energy storage capacitor 24, and has both inductive and resistive properties. Switches 32 and 34 are coupled to energy storage capacitor 24 by the bridge line 28.

Patient 16 is connected to the left side of H-bridge 14 by an apex line 17, and to the right side of H-bridge 14 by a sternum line 19. As depicted in FIG. 2, apex line 17 and sternum line 19 are connected to the patent apex and sternum electrode lines 15a and 15b, respectively, by a transfer relay circuit 35. Microprocessor 20 is connected to driver circuits 51 and 52 by control lines 42a and 42b, respectively. Microprocessor 20 is coupled by a control line 42cd to driver circuit 53a, which is coupled by a control line 42cd to driver circuits 53 and 54. Microprocessor 20 is also coupled to transfer relay circuit 35 by control line 36.

As will be described in more detail below, application of appropriate control signals by microprocessor 20 over the control lines causes switches 31-34 to be appropriately opened and closed, thereby allowing H-bridge 14 to conduct energy from energy storage capacitor 24 to patient 16 in the form of a defibrillation pulse. The operation and components H-bridge output circuit 14 are described in more detail in U.S. patent application No. 10/186,218, filed Jun. 26, 2002, and U.S. patent application No. 10/141,687, filed May 7, 2002, each of which are commonly assigned and each of which are hereby incorporated by reference in their entireties.

The defibrillator 8 also includes preamplifier ECG and impedance drive and measurement circuitry 37. The preamplifier ECG and impedance drive and measurement circuitry 37 is coupled to the patient apex and sternum lines 15a and 15b. The preamplifier ECG and impedance drive and measurement circuitry 37 is controlled by a control line 43 from the microprocessor 20 and provides measurements on a measurement line 44 to the microprocessor 20. As shown in FIG. 2, the measurement and control circuit 10, energy storage capacitor 12, output circuit 14, charging circuit 18, and preamplifier circuit 37 are all referenced to the common ground 28.

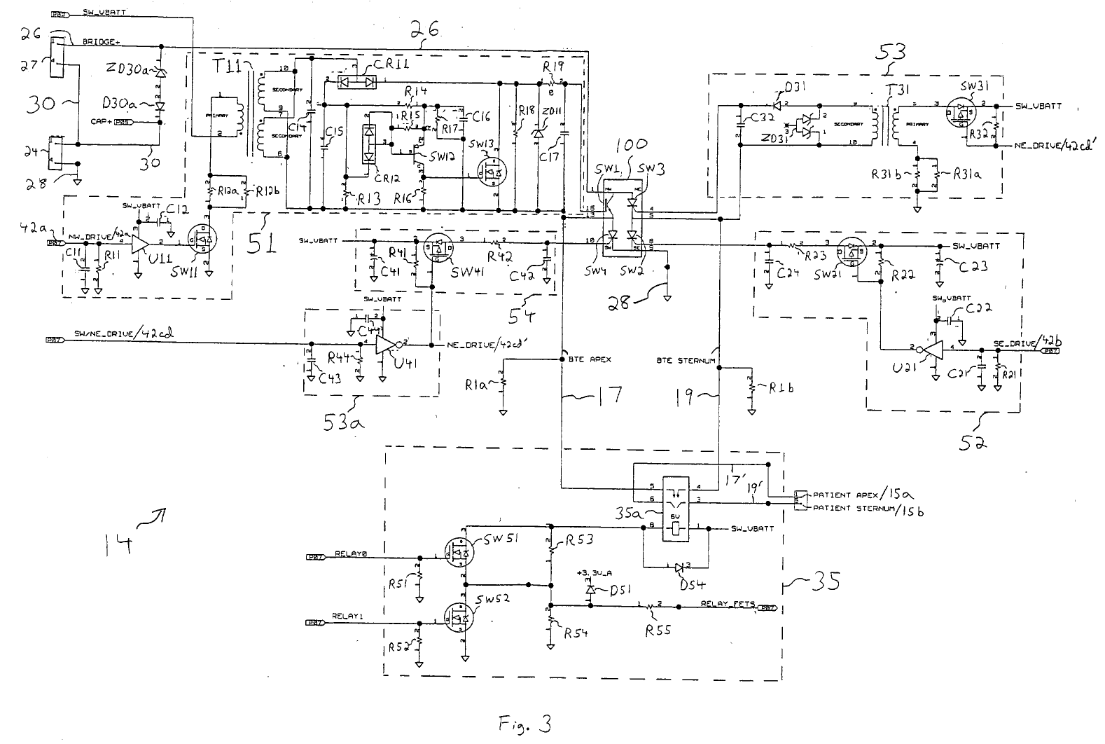

In order to simplify the block diagram of FIG. 2, the battery and power supplies circuit 39 of FIG. 1 has not been shown therein, although it will be understood that the battery and power supplies circuit 39 provides power to the circuit components as illustrated in FIG. 1. Furthermore, as described in more detail below, various power outputs from the battery and power supplies circuit 39 are coupled to circuit components as shown in the schematic diagrams of FIGS. 3, 5 and 7A-7C. For example, some of the various power outputs from the battery and power supplies circuit 39 include the voltage line SW-VBATT, the voltage line PAD+, and the voltage line PAD−.

A schematic diagram of a preferred construction of H-bridge 14 and transfer relay 35 is shown in FIG. 3. H-bridge 14 uses four output switches SW1-SW4 to conduct energy from energy storage capacitor 24 to patient 16. Switches SW1-SW4 correspond to switches 31-34 of FIG. 2, respectively. Switches SW2, SW3 and SW4 are semiconductor switches, preferably silicon controlled rectifiers (SCRs). Switch SW1 is an insulated gate bipolar transistor (IGBT). Switches SW1-SW4 can be switched from an off (nonconducting) to an on (conducting) condition.

Each of the switches SW1-SW4 is implemented as a single power switch device. Switches SW1-SW4 are packaged in a single surface-mountable package 100 for ease in manufacturing. This circuit package achieves a substantial reduction in overall parts count over previous external defibrillator H-bridges which required multiple switches in each leg, e.g., two or more IGBTs in a leg, and which were not designed to be provided in a single package. The reduction in overall parts count and ease of manufacturing of the single-surface mountable package improves the reliability and manufacturability of the external defibrillator 8. In addition, the use of the single IGBT device for a power switch in one of the legs of the H-bridge circuit simplifies the drive circuit requirements for the IGBT over previous H-bridge designs, which utilized multiple IGBT devices.

In the defibrillation mode, defibrillator 8 generates a biphasic defibrillation pulse for application to the patient 16. Initially, switches SW1-SW4 and the transfer relay 35 are opened. Charging of energy storage capacitor 24 is started, and monitored by microprocessor 20 (FIG. 2). When energy storage capacitor 24 is charged to a selected energy level and the transfer relay 35 is closed, switches SW1 and SW2 are switched on so as to connect energy storage capacitor 24 with apex line 17 and sternum line 19 for the application of a first phase of a defibrillation pulse to patient 16. The first phase of the biphasic pulse is therefore a positive pulse from the apex to the sternum of patient 16.

Before energy storage capacitor 24 is completely discharged, switch SW1 is biased off to prepare for the application of the second phase of the biphasic pulse. Once switch SW1 is biased off, switch SW2 will also become nonconducting as the current though the SCR switch SW2 drops below the holding current for the SCR.

After the end of the first phase of the biphasic defibrillation pulse, switches SW3 and SW4 are switched on to start the second phase of the biphasic pulse. Switches SW3 and SW4 provide a current path to apply a negative defibrillation pulse to patient 16. The polarity of the second phase of the defibrillation pulse is therefore opposite in polarity to the first phase of the biphasic pulse. The end of the second phase of the biphasic pulse is truncated by switching on switch SW1 and switch SW2 to provide a shorted path for the remainder of the capacitor energy through switches SW1 and SW4 and also through switches SW2 and SW3. After energy storage capacitor 24 is discharged, switches SW1-SW4 go to a nonconducting state. Patient isolation relay 35 is then opened. Energy storage capacitor 24 may then be recharged to prepare defibrillator 8 to apply another defibrillation pulse.

As described above, the four output switches SW1-SW4 can be switched from an off (nonconducting) state to an on (conducting) state by application of appropriate control signals on control lines 42a, 42b, and 42cd. In order to allow the SCRs and IGBT to conduct a range of high and low currents required for various applications, special switch driving circuits 51-54 are coupled to switches SW1-SW4, respectively. Control lines 42a and 42b, are connected to switch driving circuits 51 and 52, and control line 42cd is connected to switch driving circuit 53a, which is in turn connected by control line 42cd′ to driving circuits 53 and 54, so as to allow microprocessor 20 to control the state of the switches.

As noted above, IGBT switch SW1 is driven by switch driving circuit 51. Switch driving circuit 51 amplifies the control signal 42a and provides it to the gate of the IGBT switch SW1. It is desirable to drive the IGBT switch SW1 with a high voltage at its gate so that the switch will be able to conduct high currents, as will be described in more detail below. As will also be described in more detail below, it is also desirable to control a turn-on and turn-off time of the IGBT switch SW1 so as to ensure proper operation of the other switches within the H-bridge 14.

Switch driving circuit 51 includes resistors R11-R19, capacitors C11-C17, switches SW11-SW13, a component U11, a transformer T11, components CR11 and CR12, and a zener diode ZD11. On the primary side of the transformer T11, the control signal 42a determines the current through the transformer. Capacitor C11 and resistor R11 are coupled in parallel between the control signal line 42a and ground. The control signal line 42a is coupled to the input of component U11. The negative power supply input of component U11 is coupled to ground while the positive power supply input is coupled to the battery voltage line SW-VBATT. Capacitor C12 is coupled between the positive power supply input of component U11 and ground. The output of component U11 is coupled to the gate of switch SW11. The source of switch SW11 is coupled to ground, while the drain is coupled through the parallel resistors R12a and R12b to the non-dotted end of the primary winding of transformer T11. The dotted end of the primary winding of transformer T11 is coupled to the battery voltage line SW-VBATT. When an oscillating control signal is provided on control line 42a, switch SW11 is caused to be turned off and on, thus creating an oscillating current through the primary winding of the transformer T11, which results in a current being generated in the secondary windings of the transformer T11, as will be described in more detail below.

The secondary windings of the transformer T11 include two windings coupled in parallel. Capacitor C14 is also coupled in parallel with the secondary windings. Component CR11 is shown as being schematically represented by two diodes connected at their anodes, with three pins 1, 2, and 3, with pins 1 and 2 being connected to the cathodes of the respective two diodes and pin 3 being connected at the junction of the anodes of the two diodes. Pin 3 of the component CR11 is coupled to the dotted ends of the secondary windings of transformer T11. Capacitor C15 is coupled between pin 2 of component CR11 and the non-dotted ends of the secondary windings of the transformers T11. A resistor R13 is coupled in parallel with the capacitor C15. Component CR12 is similar to component CR11, and also includes three pins 1, 2, and 3. Pin 1 of component CR12 is coupled to pin 2 of component CR11. Pin 2 of component CR12 is coupled to pin 3 of component CR12. The base of switch SW12 is coupled to pin 3 of component CR12. Resistor R16 is coupled between the collector of switch SWl2 and the non-dotted ends of the secondary windings of transformer T11. Resistor R15 is coupled between the emitter of switch SW12 and pin 3 of component CR12. Resistor R14 is coupled between the emitter of switch SWl2 and pin 2 of component CR11. Resistor R17 and capacitor C16 are coupled in parallel between the emitter of switch SWl2 and the non-dotted end of the secondary windings of transformer T11. The collector of switch SW12 is coupled to the gate of switch SW13. The drain of switch SW13 is coupled to pin 1 of component CR11, and the source of switch SW13 is coupled to the non-dotted end of the secondary windings of the transformer T11. Resistor R18 and zener diode ZD11 are coupled in parallel between pin 1 of component CR11 and the non-dotted ends of the secondary windings of transformer T11. Resistor R19 and capacitor C17 are coupled in series between pin 1 of component CR11 and the non-dotted ends of the secondary windings of transformer T11. The circuit node between the resistor R19 and capacitor C17 is coupled to the gate of the IGBT switch SW1. As noted above, IGBT switch SW1 is coupled between the bridge line 26 and the apex line 17.

As noted above, control signal 42a determines the current through the primary winding of transformer T11. The resulting current generated in the secondary windings of transformer T11 travels through the above-described components to apply a voltage to the gate of IGBT switch SW1. The turn-on and turn-off time of IGBT switch SW1 is thus controlled, at least in part, by the above-described components which control the voltage applied to the gate of IGBT switch SW1.

It will be appreciated that transformer T11 provides isolation of the high voltage circuitry including IGBT switch SW1, from the low voltage control circuitry including control signal 42a. It will also be appreciated that the switch driving circuit 51 amplifies the control signal 42a for use in driving the gate of the IGBT switch SW1. In one embodiment, the gate of the IGBT switch SW2 may be driven with up to 30 volts.

High currents may sometimes occur in H-bridge 14. One way that high currents may be created is when low resistance is placed between the shock paddles. When this happens, a high current flows between apex line 17 and sternum line 19. In this embodiment, to accommodate high currents without damaging IGBT switch SW1, IGBT switch SW1 may be biased by a high gate voltage (e.g., 30 volts) such that the IGBT can safely conduct upwards of 200 amperes of current. When very low patient impedances are detected, the control circuitry of the defibrillator 8 limits the charge voltage so as to attempt to ensure that the defibrillator does not deliver a current of more than 200 amps to the patient.

In one embodiment, the drive circuit 51 is designed so that IGBT switch SW1 is turned on relatively slowly when compared to the fast turn-on of SCR switches SW2, SW3, and SW4. A slow turn-on for IGBT switch SW1 is desirable because the IGBT switch is on the same side of H-bridge 14 as SCR switch SW4. SCR switch SW4 is controlled by the control signal on control line 42cd, but due to the nature of SCR switches, the SCR switch may be accidentally turned on regardless of the signal on control line 42cd if a rapid voltage change occurs across SCR switch SW4. If IGBT switch SW1 was therefore turned on too quickly, the resulting rate of change of the voltage across SCR switch SW1 might cause it to turn on accidentally. In contrast to the slow turn-on of IGBT switch SW1, the turn-off of the IGBT switch may be performed relatively quickly. The IGBT switch can be quickly turned off because at turn-off there is no concern that the sensitive SCR switches will accidentally turn on.

It will be appreciated that driving circuit 51 allows the IGBT to be used in external defibrillator 8 where extremely high voltages must be switched in the presence of SCRs. The driving circuit and the use of the single IGBT switch minimizes the number of components required to switch a defibrillation pulse of 200 or more joules. In addition to conducting high currents associated with high-energy defibrillation pulses, the IGBT is also able to conduct very low currents that are associated with low energy defibrillation pulses.

It will be appreciated that the above-described circuit configuration in which the IGBT switch SW1 is placed in the northwest leg of the H-bridge is advantageous over previous prior art designs, in which the IGBT switch was placed in the southeast leg of the H-bridge 14. The placement of the IGBT switch SW1 in the northwest leg is particularly advantageous in the present circuit design due to a current path that exists from the midpoint of the H-bridge 14 through the preamp protection resistors (which in one embodiment may be 12 kohms) to ground. The amount of current flowing through this path (in one embodiment 170 mA) is negligible compared with the current delivered to the patient (in one embodiment greater than 10 amps), but is sufficient to disturb the operation of the H-bridge. More specifically, using a design in which the IGBT switch is placed in the southeast leg, the current through the preamp protection resistors (in one embodiment 170 mA) would flow through the SCRs at the top of the H-bridge 14. Once one of these SCRs was turned on, it could not be turned off until the capacitor was essentially discharged because there is no mechanism for shutting off the current through the preamp path, and as is well known in the art, once current begins flowing through an SCR, it generally cannot be turned off until the current drops below a specified level. The placement of the IGBT switch SW1 in the northwest leg allows the current through the preamp path to be shut off (along with the current through the patient) at the end of the first phase of a multiphasic defibrillation pulse.

SCR switch SW2 is driven by drive circuit 52, while SCR switch SW4 is driven by the combination of drive circuits 53a and 54. The components of drive circuit 52 and the combination of drive circuits 53a and 54 are similar. For purposes of this description, therefore, only the construction and operation of the combination of switch driving circuits 53a and 54 will be described. Those skilled in the art will recognize that the combination of switch driving circuits 53a and 54 operate in a similar manner to switch driving circuit 52. The combination of switch driving circuits 53a and 54, and the switch driving circuit 52, are designed to drive the SCR switches SW4 and SW2, respectively, so that they are both able to conduct the high-energy defibrillation pulses of 200 or more joules, as well as remaining conducting during low-energy defibrillation pulses.

Switch driving circuit 53a receives control signal 42cd and outputs control signal 42cd′. Switch driving circuit 53a includes capacitors C43 and C44, resistor R44, and component U41. Capacitor C43 and resistor R44 are coupled in parallel between the control signal line 42cd and ground. The negative power supply input of component U41 is coupled to ground, while the positive power supply input is coupled to the battery voltage line SW-VBATT. Capacitor C44 is coupled between the positive power supply input of component U41 and ground. The input of component U41 is coupled to the control signal line 42cd, while the output is coupled to the control signal line 42cd′.

Driver circuit 54 receives control signal line 42cd′, and drives SCR switch SW4. Driver circuit 54 includes capacitors C41 and C42, resistors R41 and R42, and switch SW41. Control signal line 42cd′ is coupled to the gate of switch SW41. Resistor R41 is coupled between the gate of switch SW41 and the source of switch SW41. Capacitor C41 is coupled between the source of switch SW41 and ground. The source of switch SW41 is also coupled to the battery voltage line SW-VBATT. Resistor R42 is coupled between the drain of switch SW41 and the gate of SCR switch SW4. Capacitor C42 is coupled between the gate of SCR switch SW4 and ground.

It will be appreciated that the combination of the above-described driver circuits 53a and 54 are generally driven by the control signal lines 42cd and 42cd′, which as will be described in more detail below may in one embodiment carry an oscillating drive signal. It will be understood that in other embodiments, the SCR switch SW4 may be driven by a DC gate drive signal, similar to the one used on control line 42b for SCR switch SW2. It will be appreciated that the driver circuit 52 may be advantageously used in combination with a DC gate drive signal that is applied to the lower SCR switch SW2. The benefits of this design can be illustrated by comparison with certain prior art defibrillators, which isolated the high-voltage H-bridge from the low-voltage control circuit ground potential. This type of prior art configuration required a transformer to couple a drive signal to the SCR gate, which consequently required the gate to be driven with AC signals, in that transformers are unable to pass DC signals. The utilization of a common ground for the high-voltage and low-voltage circuits in the present invention allows the gate of the SCR switch SW2 to be driven directly from FET switch SW21 with a DC signal.

SCR switch SW3 is driven by drive circuit 53. Drive circuit 53 includes a transformer T31 for isolating the high-voltage SCR switch SW3 from the low-voltage control circuitry. The isolation of the gate drive allows for the use of small, low voltage parts, in contrast to the relatively high-voltage gate drive components that would be required if the gate drive was not isolated. Switch driving circuit 53 is designed to drive the SCR switch SW3 so that it is able to both conduct the high-energy defibrillation pulses of 200 or more joules as well as remaining conducting during lower energy defibrillation pulses.

Switch driving circuit 53 receives the control signal line 42cd′ from switch driving circuit 53a, and drives SCR switch SW3. Switch driving circuit 53 includes switch SW31, resistors R31a, R31b, R32, transformer T31, diode D31, capacitor C32, and component ZD31. The gate of switch SW31 receives the control signal line 42cd′. Resistor R32 is coupled between the source of switch SW31 and the gate of switch SW31. The source of switch SW31 receives the battery voltage line SW-VBATT. The drain of switch SW31 is coupled to the dotted end of the primary winding of transformer T31. Resistors R31a and R31b are coupled in parallel between the non-dotted end of the primary winding of transformer T31 and ground. Component ZD31 is coupled in parallel with the secondary winding of transformer T31. The anode of diode D31 is coupled to the dotted end of the secondary winding of transformer T31, while the cathode is coupled to the gate of SCR switch SW3. Capacitor C32 is coupled between the gate of SCR switch SW3 and sternum line 19. The non-dotted end of the secondary winding of transformer T31 is coupled to sternum line 19.

On the secondary winding side of transformer T31, the anode of diode D31 is connected to the dotted end of the secondary winding of transformer T31, and the cathode of diode D31 is coupled to the gate of SCR switch SW3. Capacitor C32 is coupled between the cathode of diode D31 and sternum line 19. Sternum line 19 is coupled to the non-dotted end of the secondary winding of transformer T31. Component ZD31 is coupled between the dotted and non-dotted ends of the secondary winding of transformer T31. As noted above, the anode of SCR switch SW3 is coupled to the bridge line 26, while the cathode is coupled to sternum line 19.

To turn on switch SW3, an oscillating control signal is provided on control line 42cd. In this embodiment, the oscillating control signal may be a pulse train. In one embodiment the pulse train control signal on control line 42cd′ is provided as a series of 10 pulses, the pulses being 1 microsecond wide and being provided every 2.5 microseconds. The pulse train control signal repeatedly turns control switch SW31 on and off, producing a changing voltage across the primary winding of transformer T31. The voltage is stepped down by transformer T31 and rectified by diode D31 before being applied to the gate of SCR switch SW3. In one embodiment, a 10% duty cycle pulse train on the control line 42cd has been found to be adequate to maintain SCR switch SW3 in a conducting state. As long as the control signal is applied to the switch driving circuit 53, the switch SW3 will generally remain in the conducting state. The switch SW3 remains in the conducting state even when conducting relatively low defibrillation currents. As is well known, once triggered or latched on, an SCR generally remains in the conducting state until the current through the SCR drops below a minimum level (e.g., 90 mA), even if the gate voltage of the SCR is grounded.

Protection for the switches SW1-SW4 is provided in part by protective component 27, which has both inductive and resistive properties. Protective component 27 is coupled between bridge lines 26 and 30. In one embodiment, protective component 27 is implemented with a coil of resistance wire that provides an inductive resistance. Protective component 27 limits the rate of change of the voltage across, and current flow to, SCR switches SW2, SW3, and SW4. Too high of a rate of change of the voltage across an SCR switch is undesirable because it can cause the SCR switch to inadvertently turn on. For example, since SCR switches SW2 and SW3 are on the same side of H-bridge 14, any time SCR switch SW3 is abruptly turned on, a rapid voltage change may also result across SCR switch SW2. To prevent rapid voltage changes, protective component 27 reduces the rate of change of the voltage across SCR switch SW2 when SCR switch SW3 is turned on. Also, too high of a current flow can damage the switches SW2, SW3, and SW4, and protective component 27 limits the current flow in H-bridge 14. The use of protective component 27 therefore reduces the need for additional protective components that would otherwise need to be coupled to switches SW2, SW3, and SW4.

The H-bridge 14 also includes resistors R1a and R1b. Resistor R1a is coupled between apex line 17 and ground. Resistor R1b is coupled between sternum line 19 and ground.

It will be appreciated that one advantage of H-bridge 14 described above is that it allows external defibrillator 8 to generate and apply a high-energy biphasic waveform to a patient. For prior defibrillators providing a monophasic waveform, the standard energy level in the industry for the discharge has been equal to or greater than 200 joules. The above described circuit allows the same amount of energy (approximately equal to or greater than 200 joules) to be delivered to the patient in a biphasic waveform, thereby resulting in a greater certainty of defibrillation effectiveness for a broader range of patients. At the same time, the circuit incorporates special driving circuitry to allow even very low energy biphasic waveforms to be delivered to the patient.

FIG. 3 also shows transfer relay 35, which includes a relay 35a which is driven by drive signals RELAY0 and RELAY1. As illustrated in FIG. 3, relay 35a has pins 1, 3, and 4 on its right side, and pins 5, 6, and 8 on its left side. Pin 4 is coupled to sternum line 19, which when the relay is closed is connected to pin 3, which is coupled to patient sternum line 19′, which is coupled to patient sternum electrode line 15b. Pin 5 is coupled to apex line 17, which when the relay is closed is connected to pin 6, which is coupled to patient apex line 17′, which is coupled to patient apex electrode line 15a. Pin 1 is coupled to the battery voltage line SW-VBATT. It will be appreciated that in some embodiments, the patient apex line 17′ and the patient apex electrode line 15a may actually be the same line, as may also be the case with the patient sternum line 19′ and the patient sternum electrode line 15b.

Relay 35 also includes driving circuitry for driving relay 35a, which includes resistors R51-R55, switches SW51 and SW52, and diodes D51 and D54. The anode of diode D54 is coupled to pin 8 of relay 35a, while the cathode of diode D54 is coupled to pin 1 of relay 35a. The drain of switch SW51 is coupled to pin 8 of relay 35a. The gate of switch SW51 is coupled to the drive signal RELAY0. Resistor R51 is coupled between the gate of switch SW51 and ground. The source of switch SW51 is coupled to the drain of switch SW52. The gate of switch SW52 is coupled to the drive signal RELAY1. Resistor R52 is coupled between the gate of switch SW52 and ground. The source of SW52 is coupled to ground. Resistor R53 is coupled between the drain of switch SW51 and the source of SW51. Resistor R54 is coupled between the drain of switch SW52 and ground. The anode of diode D51 is coupled to the drain of switch SW52, and the cathode of diode D51 is coupled to the voltage line +3.3V-A. Resistor R55 is coupled between the drain of switch SW52 and the control signal line RELAY-FETS.

Transfer relay 35 is operated such that when the defibrillator 8 is to apply a defibrillation pulse to a patient 16, the relay 35a is closed. When the relay 35a is open, it isolates the patient 16 from the rest of the defibrillator 8 circuitry. As described above, the transfer relay 35 includes drive circuitry for driving the relay 35a. The drive circuitry is controlled by the control signal lines RELAY0, RELAY1, and RELAY-FETS.

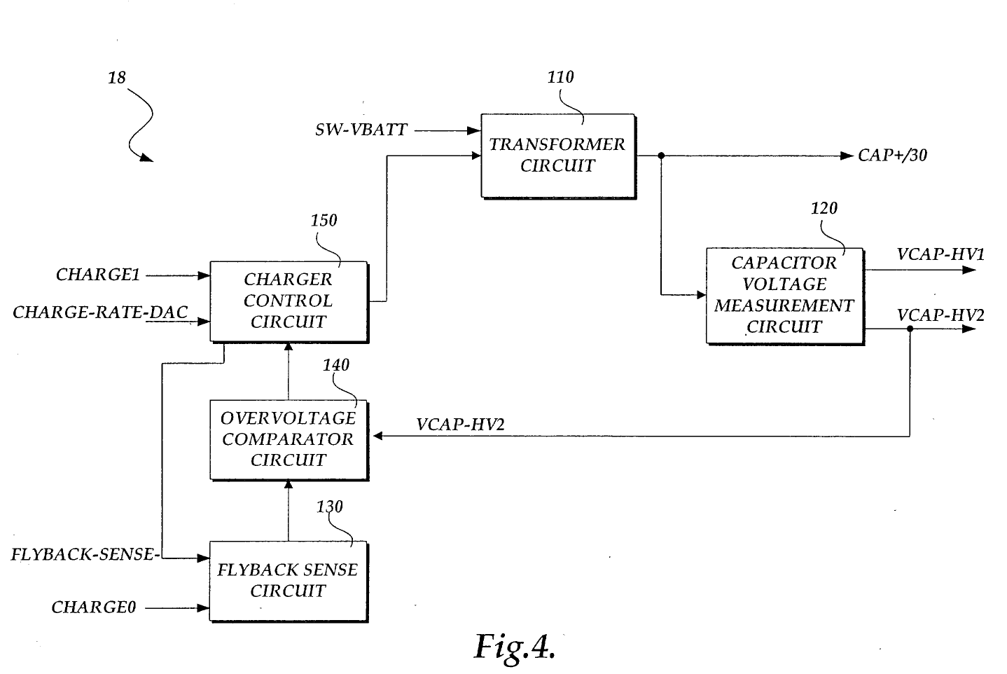

FIG. 4 is a block diagram of the charging circuit 18 that is used to charge the energy storage capacitor 24. As described above, the charging circuit 18 is referenced to the same common ground 28 as the output circuit 14 and the preamplifier circuit 37. As described above, the measurement and control circuit 10 controls the charging circuit 18 to charge the energy storage capacitor to a desired voltage level. After charging to a desired level, the energy stored in the energy storage capacitor may be delivered to the patient 16 in the form of a defibrillation pulse.

As shown in FIG. 4, the charging circuit 18 includes a transformer circuit 110, a capacitor voltage measurement circuit 120, a flyback sense circuit 130, an overvoltage comparator circuit 140, and a charger control circuit 150. Transformer circuit 110 receives the battery voltage line SW-VBATT and provides a stepped-up voltage on the voltage line CAP+. This stepped up voltage on the voltage line CAP+ is used to charge the energy storage capacitor 24 (FIG. 2), and is also provided as an input to the capacitor voltage measurement circuit 120. The capacitor voltage measurement circuit 120 provides two output signal lines VCAP-HV1 and VCAP-HV2, which represent the voltage on the energy storage capacitor 24, and provides the measurements to the control circuit for the defibrillator 8. The signal line VCAP-HV2 is also coupled as an input to the overvoltage comparator circuit 140. Overvoltage comparator circuit 140 also receives a control signal from the flyback-sense circuit 130.

Flyback-sense circuit 130 receives a control signal line CHARGE0, and also a control signal line FLYBACK-SENSE from the charger control circuit 150. Flyback sense circuit 130 inhibits the signal on control signal line FLYBACK-SENSE for a specified time period (e.g., 40 milliseconds) after the signal on the control signal line CHARGE0 goes high. The overvoltage comparator circuit 140 receives the measurement signal line VCAP-HV2, as well as the control signal from the flyback sense circuit 130. The overvoltage comparator circuit 140 provides an output to the charger control circuit 150. Charger control circuit 150 receives control signal lines CHARGE1 and CHARGE-RATE-DAC, and outputs a control signal to control the charging of the transformer 110.

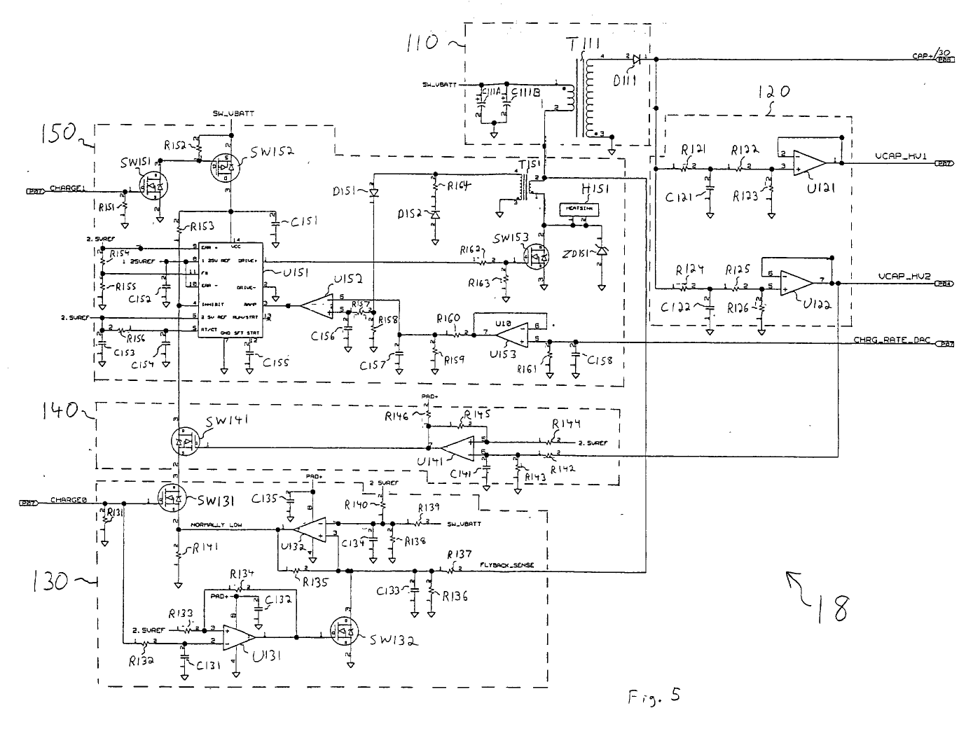

FIG. 5 is a schematic diagram of a preferred embodiment of the charging circuit 18 of FIG. 4. As illustrated in FIG. 5, the transformer circuit 110 includes a transformer T111, a diode D111, and capacitors C111A and C111B. In one embodiment, the transformer T111 is able to step, up the voltage on the battery voltage line SW-VBATT to 2300 volts. The dotted end of the primary winding of the transformer T111 is connected to the battery voltage line SW-VBATT. Capacitors C111A and C111B are coupled in parallel between the battery voltage line SW-VBATT and ground. The dotted end of the secondary winding of transformer T111 is coupled to ground, while the non-dotted end of the secondary winding of transformer T111 is coupled to the anode of diode D111. The cathode of diode D111 is coupled to the positive terminal of the energy storage capacitor through the charging line CAP+/30 (referenced as line 30 in FIG. 2).

The charging line CAP+/30 is coupled to the capacitor voltage measurement circuit 120. The capacitor voltage measurement circuit 120 includes resistors R121 to R126, capacitors C121 and C122, and components U121 and U122. The resistor/capacitor structure for the components U121 and U122 are similar, therefore only the structure for component U121 will be described herein. The negative input of component U121 is coupled to the output of the component U121. The positive input of the component U121 is coupled through resistor R123 to ground. One side of resistor R122 is coupled to the positive input of the component U121, while the other side of resistor R122 is coupled to a circuit node between resistor R121 and capacitor C121. Capacitor C121 is coupled between the circuit node and ground, while resistor R121 is coupled between the circuit node and the capacitor charging line CAP+/30.

In one embodiment, the sizes of resistors R121-R123 and capacitor C121 are selected so that the gain of component U121 is 1/735, while the sizes of the components R124-R126 and C122 are selected so that the gain of the component U122 is 1/592. The output of the component U121 is provided on the measurement line VCAP-HV1, while the output of the component U122 is provided on the measurement line VCAP-HV2. The measurement line VCAP-HV2 is also coupled to the overvoltage comparator circuit 140. As described above, overvoltage comparator circuit 140 also receives a control signal from flyback sense circuit 130.

Flyback sense circuit 130 includes resistors R131-R141, capacitors C131-C135, switches SW131 and SW132, and components U131 and U132. The signal line CHARGE0 is coupled to the gate of switch SW131. Resistor R131 is coupled between the gate of switch SW131 and ground. The drain of switch SW131 is coupled to the overvoltage comparator circuit 140, while the source of switch SW131 is coupled through resistor R141 to ground. The gate of switch SW131 is coupled through resistor R132 to the negative input of component U131. The negative input of component U131 is also coupled through capacitor C131 to ground. The negative power supply input of component U131 is coupled to ground, while the positive power supply input of component U131 is coupled to the voltage line PAD+. Capacitor C132 is coupled between the positive power supply input of component U131 and ground. Resistor R133 is coupled between a voltage line 2.5-VREF and the positive input of the component U131. Resistor R134 is coupled between the positive input of component U131 and the output of component U131. The output of component U131 is coupled to the gate of switch SW132.

The source of switch SW132 is coupled to ground, while the drain is coupled to the positive input of component U132. Capacitor C133 and resistor R136 are coupled in parallel between the drain of switch SW132 and ground. Resistor R137 is coupled between the drain of switch SW132 and the signal line FLYBACK-SENSE from control charger circuit 150. Resistor R135 is coupled between the positive input of component U132 and the output of component U132. The negative power supply input of component U132 is coupled to ground, while the positive power supply input is coupled to the voltage line PAD+. Capacitor C135 is coupled between the positive power supply input of the component U132 and ground.

Capacitor C134 and resistor R138 are coupled in parallel between the negative input of component U132 and ground. Resistor R140 is coupled between the negative input of component U132 and the voltage line 2.5-VREF. Resistor R139 is coupled between the battery voltage line SW-VBATT, and the resistor R138. The output of component U132 is coupled to the source of switch SW131. Resistor 141 is coupled between the source of switch SW131 and ground. The drain of switch SW131 is coupled to the overvoltage comparator circuit 140.

Overvoltage comparator circuit 140 includes resistors R142-R146, capacitors C141, component U141, and switch SW141. The source of switch SW141 is coupled to the drain of switch SW131 of the flyback sense circuit 130. The drain of switch SW141 is coupled to the charger control circuit 150. The gate of switch SW141 is coupled to the output of component U141. Resistor R142 is coupled between the measurement line VCAP-HV2 and the negative input of the component U141. Resistor R143 and capacitor C141 are coupled in parallel between the negative input of the component U141 and ground. Resistor R144 is coupled between voltage line 2.5-VREF and the positive input of component U141. Resistor R145 is coupled between the positive input of component U141 and the output of component U141. Resistor R146 is coupled between the output of component U141 and the voltage line PAD+. In one embodiment, the overvoltage comparator circuit 140 operates to cut off the charging voltage for the capacitor at 2300 volts.

Charger control circuit 150 receives a signal from the drain of switch SW141 in the overvoltage comparator circuit 140. Charger control circuit 150 includes resistors R151-R164, capacitors C151-C158, switches SW151-SW153, diodes D151 and D152, components U151-U153, heat sink H151, and zener diode ZD151. The input signal line CHARGE1 is coupled to the gate of switch SW151. Resistor R151 is coupled between the gate of switch SW151 and ground. The source of switch SW151 is coupled to ground, while the drain of switch SW151 is coupled to the gate of switch SW152. Resistor R152 is coupled between the gate of switch SW152 and the source of switch SW152. The source of switch SW152 is coupled to the battery voltage line SW-VBATT. The drain of switch SW152 is coupled to the VCC pin 14 of component U151.

Component U151 has 14 pins, including a DRIVE+ pin 1, a DRIVE− pin 2, a RAMP pin 3, an INHIBIT pin 4, an RT/CT pin 5, a 2.5 V-REF pin 6, a GND pin 7, a 1.25 V-REF pin 8, an ERR+ pin 9, an ERR− pin 10, an FB pin 11, an SFT-STRT pin 12, a RUN/STRT pin 13, and a VCC pin 14. DRIVE+ pin 1 is coupled through resistor R162 to the gate of switch SW153. DRIVE− pin 2 is coupled to ground. RAMP pin 3 is coupled to the output of component U152. INHIBIT pin 4 is coupled through resistor R153 to VCC pin 14. INHIBIT pin 4 is also coupled to the drain of switch SW151 from the overvoltage comparator circuit 140. RT/CT pin 5 is coupled through capacitor C154 to ground, and is also coupled through resistor R156 to the reference voltage line 2.5-VREF. Capacitor C153 is coupled between the reference voltage line 2.5-VREF and ground. 2.5 V-REF pin 6 is coupled to the reference voltage line 2.5-VREF. GND pin 7 is coupled to ground. 1.25 V-REF pin 8 is coupled to the reference voltage line 1.25-VREF. Capacitor C152 is coupled between the reference voltage line 1.25-VREF and ground. ERR+ pin 9 is coupled to the reference voltage line 2.5-VREF and is also coupled through a resistor R154 to FB pin 11. FB pin 11 is coupled through resistor R155 to ground. ERR−+pin 10 is coupled to pin 8. SFT-STRT pin 12 is coupled through capacitor C155 to ground.

As noted above, DRIVE+ pin 1 of component U151 is coupled by resistor R162 to the gate of switch SW153. Resistor R163 is coupled between the gate of switch SW153 and ground. The source of switch SW153 is coupled to ground, while the drain is coupled to the dotted end of the primary winding of transformer T151. Heat sink H151 is coupled between the dotted end of the primary winding of transformer T151 and the cathode of zener diode ZD151. The anode of zener diode ZD151 is coupled to ground. The non-dotted end of the primary winding of transformer T151 is coupled to the non-dotted end of the primary winding of transformer T111 of transformer circuit 110.

The dotted end of the secondary winding of the transformer T151 is coupled to ground. The anode of diode D152 is coupled to ground, while the cathode is coupled through resistor R164 to the non-dotted end of the secondary winding of transformer T151. The anode of diode D151 is coupled to the non-dotted end of the secondary winding of the transformer T151. The cathode of diode D151 is coupled through resistor R157 to the positive input of component U152. The cathode of diode D151 is also coupled through resistor R158 to ground. The positive input of the component U152 is also coupled through capacitor C156 to ground.

As described above, the output of the component U152 is coupled to RAMP pin 3 of component U151. Capacitor C157 and resistor R159 are coupled in parallel between the negative input of component U152 and ground. Resistor R160 is coupled between the negative input of component U152 and the output of component U153. The negative input of component U153 is coupled to the output of component U153. Resistor R161 and capacitor C158 are coupled in parallel between the positive input of the component U153 and ground. The positive input of component U153 is also coupled to the control signal line CHRG-RATE-DAC.

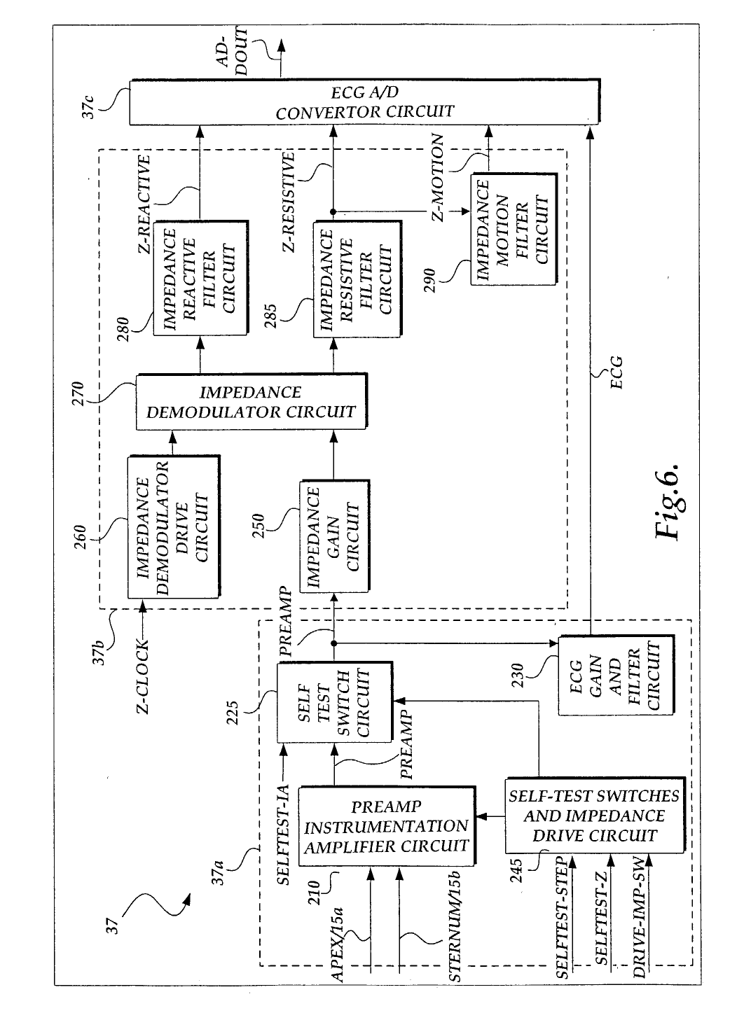

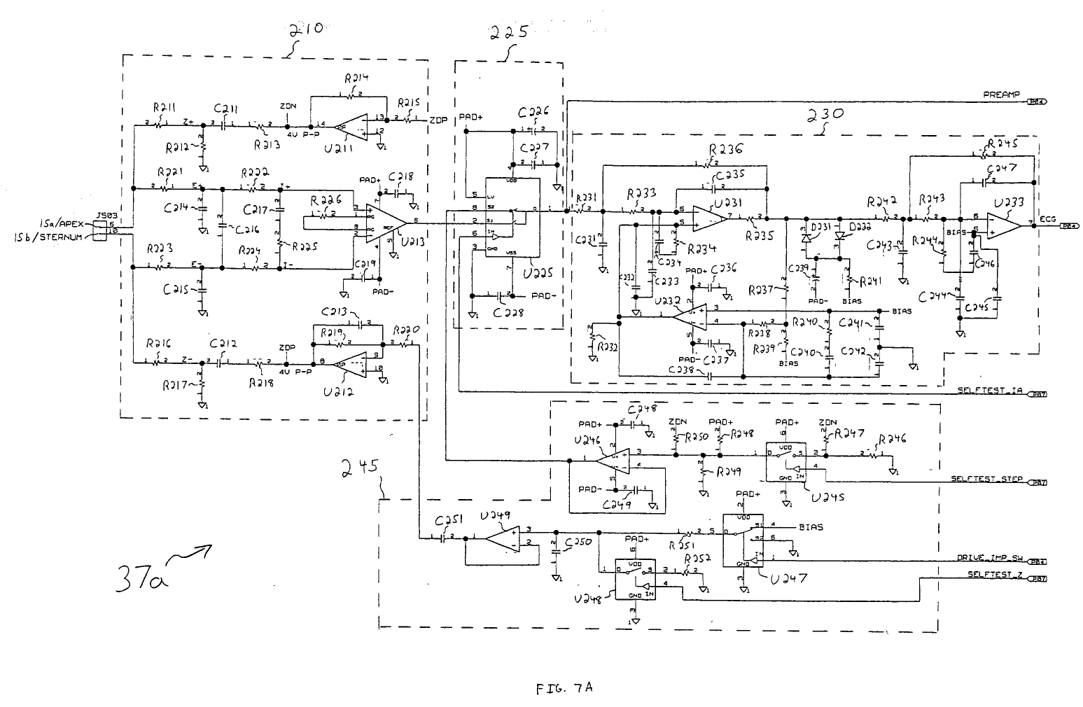

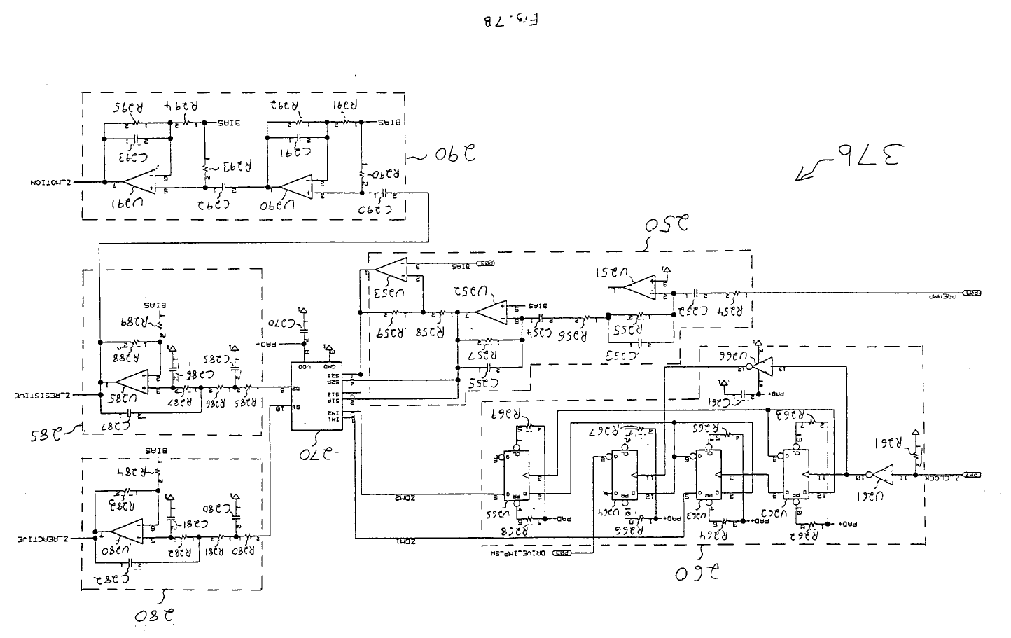

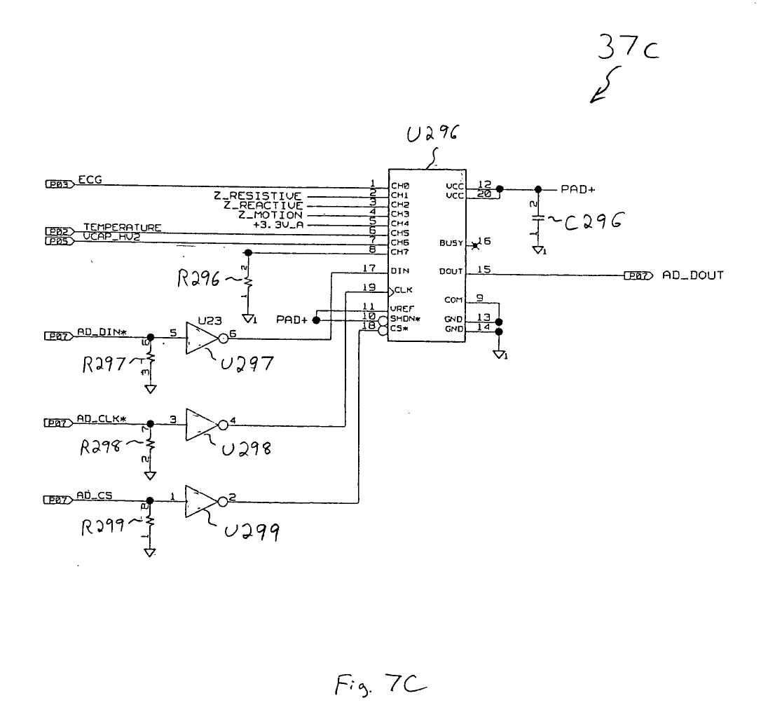

FIG. 6 is a block diagram of the preamplifier ECG and impedance drive and measurement circuit 37 of FIG. 2. As shown in FIG. 6, the preamplifier ECG and impedance drive and measurement circuit 37 includes an ECG preamp circuit 37a, an impedance demodulator circuit 37b, and an ECG A-to-D converter circuit 37c. A preferred embodiment of the ECG preamplifier circuit 37a will be described in more detail below with reference to the schematic diagram shown in FIG. 7A, while a preferred embodiment of the impedance demodulator circuit 37b will be described in more detail below with reference to the schematic diagram shown in FIG. 7B, and a preferred embodiment of the ECG A-to-D converter circuit 37c will be described in more detail below with reference to the schematic diagram shown in FIG. 7C.

As shown in FIG. 6, the ECG preamplifier circuit 37a includes a preamp instrumentation amplifier 210, a self test switch 225, an ECG gain and filter circuit 230, and a self test switches and impedance drive circuit 245. The preamp instrumentation amplifier circuit 210 measures the apex and sternum voltages over the lines APEX/15a and STERNUM/l 5b. Preamp instrumentation amplifier circuit 210 outputs a signal on the signal line PREAMP. Self test switch circuit 225 passes the signal on the signal line PREAMP depending on the control signal from the self test switches and impedance drive circuit 245 and the control signal line SELFTEST-IA. Self test switches and impedance drive circuit 245 receives control signals on signal lines SELFTEST-STEP, SELFTEST-Z, and DRIVE-IMP-SW. The ECG gain and filter circuit 230 receives the signal line PREAMP and provides an output on the signal line ECG. The ECG gain and filter circuit 230 includes low frequency filter and gain stages for processing the information on the signal line PREAMP for ECG signals.

The impedance demodulator circuit 37b includes the impedance gain circuit 250, the impedance demodulator drive circuit 260, the impedance demodulator circuit 270, the impedance reactive filter circuit 280, the impedance resistive filter circuit 285, and the impedance motion filter circuit 290. In general, the impedance demodulator circuit 37b utilizes high frequency filter and gain stages to process the information received on the signal line PREAMP to determine relevant impedance information. As shown in FIG. 6, the impedance gain circuit 250 receives the signal line PREAMP and outputs a signal to the impedance demodulator circuit 270. Impedance demodulator circuit 270 is controlled in part by impedance demodulator drive circuit 260, which receives a clock signal on the signal line Z-CLOCK. Impedance demodulator circuit 270 outputs a first signal to the impedance reactive filter circuit 280, and a second signal to the impedance resistive filter circuit 285. The impedance reactive filter circuit 280 outputs a signal line Z-REACTIVE, while the impedance resistive filter circuit 285 outputs a signal line Z-RESISTIVE. The impedance motion filter circuit 290 receives the signal line Z-RESISTIVE as an input, and outputs a signal line Z-MOTION. The ECG A-to-D converter circuit 37c receives the signal lines Z-REACTIVE, Z-RESISTIVE, Z-MOTION, and ECG, and outputs a signal line AD-DOUT.

FIG. 7A is a schematic diagram of a preferred embodiment of the ECG preamp circuit 37a of FIG. 6. As shown in FIG. 7A, the preamp instrumentation amplifer circuit 210 includes resistors R211-R226, capacitors C211-C219, and components U211-U213. Resistor R211 is coupled between the patient apex signal line 15a/APEX and a circuit node with the capacitor C211, which is also coupled through resistor R212 to ground. The other side of capacitor C211 is coupled through resistor R213 to the output of component U211. The output of component U211 is coupled through resistor R214 to the negative input of component U211. The negative input of component U211 is also coupled through resistor R215 to a voltage line ZDP. The positive input of component U211 is coupled to ground.

Resistor R216 is coupled between the patient sternum line 15b/STERNUM and a circuit node with the capacitor C212, which is also coupled through a resistor R217 to ground. The other side of capacitor C212 is coupled through resistor R218 to the output of component U211. Capacitor C213 and resistor R219 are coupled in parallel between the output of component U211 and the negative input of component U211. The negative input of component U211 is also coupled through resistor R220 to the self test switches and impedance drive circuit 245. The positive input of component U212 is coupled to ground.

Resistor R221 is coupled between the patient apex line 15a/APEX and a circuit node with capacitor C214, capacitor C216, and resistor R222. The other side of capacitor C214 is coupled to ground, while the other side of capacitor C216 is coupled to a circuit node with resistor R223, capacitor C215, and resistor R224, and the other side of resistor R222 is coupled to the positive input of component U213. Resistor R223 is coupled between the patient sternum line 15b/STERNUM and the circuit node with capacitor C216. The other side of capacitor C215 is coupled to ground while the other side of resistor 224 is coupled to the negative input of component U213. Capacitor C217 and resistor R225 are coupled in series between the positive input of the component U213 and the negative input of the component U213.

Resistor R226 is coupled between a first input RG of the component U213 and a second input RG of the component U213. The positive power supply of component U213 is coupled to the voltage line PAD+, which is also coupled through capacitor C218 to ground. The negative power voltage line of the component U213 is coupled to the voltage line PAD−, which is also coupled through capacitor C219 to ground. The reference input REF of component U213 is coupled to ground. The output of component U213 is coupled to the self test switch circuit 225.

Self test switch circuit 225 includes capacitors C226-C228 and a component U225. Component U225 has eight pins, including a D pin 1, a S1 pin 2, a GND pin 3, a VDD pin 4, an LV pin 5, an IN pin 6, a VSS pin 7, and an S2 pin 8. D pin 1 provides the signals on signal line PREAMP and is coupled to both the ECG gain and filter circuit 230 and to the impedance gain circuit 250 (FIG. 7B). S1 pin 2 is coupled to the output of component U213 of the preamp instrumentation amplifier circuit 210. GND pin 3 is coupled to ground, and is also coupled through capacitor C228 to VSS pin 7. VSS pin 7 is also coupled to the voltage line PAD−. VDD pin 4 is coupled to the voltage line PAD+. Capacitor C226 and C227 are coupled in parallel between VDD pin 4 and ground. LV pin 5 is coupled to the voltage line PAD+. IN pin 6 is coupled to the control signal line SELFTEST-IA. S2 pin 8 is coupled to the self test switches and impedance drive circuit 245.

The ECG gain and filter circuit 230 includes resistors R231-R245, capacitors C231-C247, and components U231-U233. Resistor R231 is coupled between D pin 1 of component U225 of self test switch 225 and a circuit node with resistor R236, resistor R233, and capacitor C231. The other side of capacitor C231 is coupled to ground, while the other side of resistor R233 is coupled to the negative input of component U231, and the other side of resistor R236 is coupled to a circuit node with capacitor C235, resistor R235, resistor R237, the cathode of diode D31, the anode of diode D32, and resistor R242. The other side of capacitor C235 is coupled to the negative input of component U231, while the other side of resistor R235 is coupled to the output of component U231, and the other side of resistor R237 is coupled to a circuit node with resistor R238 and resistor R239, and the other side of resistor R242 is coupled to a circuit node with resistor R243, resistor R245, and capacitor C243. The anode of diode D231 is coupled to the cathode of diode D232. Capacitor C239 is coupled between the anode of diode D231 and the voltage line PAD−, while the cathode of diode D232 is coupled through resistor R241 to the voltage line BIAS.

From the circuit node between the resistor R242, the resistor R243, the resistor R245, and the capacitor U243, the other side of the resistor R243 is coupled to the negative input of component R233, while the other side of resistor R245 is coupled to the output of component U233, and the other side of capacitor C243 is coupled to ground. Resistor R244 and capacitor C246 are coupled in series between the negative input of component U233 and the positive input of component U233. The positive input of component U233 is also coupled to the voltage line BIAS. Capacitor C247 is coupled between the negative input of component U233 and the output of component U233. The positive input of component U233 is coupled through capacitor C245 to ground. The negative input of component U233 is coupled through capacitor C244 to ground.

The positive input of component U232 is coupled to the voltage line BIAS. Capacitor C241 is coupled between the positive input of component U232 and ground, while capacitor C242 is coupled between the negative input of component U232 and ground. Resistor R240 and capacitor C240 are coupled in series between the positive input of component U232 and the negative input of component U232. With regard to the circuit node between resistors R237, R238, and R239, the other side of resistor R238 is coupled to the negative input of component U232, while the other side of resistor R239 is coupled to the voltage line BIAS. The negative input of component U232 is coupled through capacitor C238 to the output of component U232. The output of component U232 is coupled through resistor R232 to ground.

The positive power supply input of component U232 is coupled to the voltage line PAD+, while the negative power supply input of component U232 is coupled to the voltage line PAD−. Capacitor C236 is coupled between the positive power supply input for component U232 and ground, while capacitor C237 is coupled between the negative power supply input for component U232 and ground. Capacitor C234 and resistor R234 are coupled in series between the negative input of component U231 and the positive input of component U231. Capacitor C232 is coupled between the positive input of component U231 and ground, while capacitor C233 is coupled between the negative input of component U231 and ground.

Self test switches and impedance drive circuit 245 includes resistors R246-R252, capacitors C248-C251, and components U245-U249. Component U245 has five connected pins, including a D pin 1, a S pin 2, a GND pin 3, an IN pin 4, and a VDD pin 6. S pin 2 is coupled through resistor R246 to ground, and is also coupled through resistor R247 to the voltage line VDN. IN pin 4 is coupled to the control signal line SELFTEST-STEP. GND pin 3 is coupled to ground. VDD pin 6 is coupled to the voltage line PAD+. D pin 1 is coupled to the positive input of component U246.

Resistor R248 is coupled between the positive input of component U246 and the voltage line PAD+, while resistor R249 is coupled between the positive input of component U246 and ground, and resistor R250 is coupled between the positive input of component U246 and the voltage ZDN. The negative input of component U246 is coupled to the output of component U246. The positive power supply input of component U246 is coupled to the voltage line PAD+, while the negative power supply input of component U246 is coupled to the voltage line PAD−. Capacitor C248 is coupled between the positive voltage line input of component U246 and ground, while capacitor C249 is coupled between the negative power supply input of component U246 and ground. The output of component U246 is coupled to the S2 pin 8 of component U225 of self test switch 225.

Component U247 has six connected pins, including an N pin 1, a VDD pin 2, a GND pin 3, a S1 pin 4, a D pin 5, and a S2 pin 6. N pin 1 is coupled to the control signal line DRIVE-IMP-SW. VDD pin 2 is coupled to the voltage line PAD+. GND pin 3 is coupled to ground. S1 pin 4 is coupled to the voltage line BIAS. D pin 5 is coupled through resistor R251 to the positive input of component U249. S2 pin 6 is coupled to ground.

Component U248 has five connected pins, including a D pin 1, an S pin 2, a GND pin 3, an N pin 4, and a VDD pin 6. N pin 4 is coupled to the control signal line SELFTEST-Z. S pin 2 is coupled through resistor R252 to ground. GND pin 3 is coupled to ground. VDD pin 6 is coupled to the voltage line PAD+. D pin 1 is coupled to the positive input of component U249. The positive input of component U249 is coupled through capacitor C250 to ground. The negative input of component U249 is coupled to the output of component U249. Capacitor C251 is coupled between the output of component U249 and a circuit node with resistor R220 of the preamp instrumentation amplifier circuit 210. As described above, the other side of resistor R220 is coupled to the negative input of component U212.

With regard to the above-described components in the schematic diagram of FIG. 7A, it will be understood that certain of the components are included primarily for self test purposes. For example, capacitors C226-C228, components U245-U246, capacitors C248-C249, resistors R246-R250, component U248, and resistor R252 are included only for self test purposes. Component U225 is also included only for self test purposes, although its removal would require adding a line between pin 6 of component U213 and resistor R231.

FIG. 7B is a schematic diagram of the impedance demodulator circuit 37b of FIG. 6. As shown in FIG. 7B, the impedance demodulator drive circuit 260 includes resistors R261-R269, components U261-U266, and a capacitor C261. Signal line Z-CLOCK is coupled to the input of component U261. The input of component U261 is also coupled through resistor R261 ground. The output of component U261 is coupled to an input pin 11 of component U262.

Component U262 has six connected pins including a Q pin 8, a Q pin 9, a PR pin 10, an input pin 11, a D pin 12, and a CL pin 13. Q pin 8 is coupled to D pin 12, and is also coupled to pin 3 of component U265. Q pin 9 is coupled to input pin 3 of component U263. PR pin 10 is coupled by resistor R262 to the voltage line PAD+. CL pin 13 is coupled by resistor R263 to the voltage line PAD+.

Component U263 has six connected pins including a CL pin 1, a D pin 2, an input pin 3, a PR pin 4, a Q pin 5, and a Q pin 6. CL pin 1 is coupled by resistor 265 to the voltage line PAD+. D pin 2 is coupled to Q pin 6, and is also coupled to pin 2 of component U265. Input pin 3 is coupled to pin 9 of component U262. PR pin 4 is coupled by resistor R264 to the voltage line PAD+. Q pin 5 is coupled by signal line ZDM1 to pin 1 of component 270.

Component U264 has five connected pins including a Q pin 8, a PR pin 10, an input pin 11, a D pin 12, and a CL pin 13. Q pin 8 is coupled to the signal line DRIVE−IMP−SW. PR pin 10 is coupled by resistor R266 to the voltage line PAD+. Input pin 11 is coupled to the output of component U266. CL pin 13 is coupled by resistor R267 to the voltage line PAD+.

Component U265 has five connected pins, including a CL pin 1, a D pin 2, an input pin 3, a PR pin 4, and a Q pin 5. CL pin 1 is coupled by resistor R269 to the voltage line PAD+. D pin 2 is coupled to the circuit node between pin 12 of component U264, pin 6 of component U263, and pin 2 of component U263. Input pin 3 is coupled to the circuit node between pin 8 of component U262 and pin 12 of component U262. PR pin 4 is coupled by resistor R268 to the voltage line PAD+. Q pin 5 is coupled by signal line ZDM2 to pin 5 of component 270.

The input of component 266 is coupled to the output of component U261. The positive power supply input of component U266 is coupled to the voltage line PAD+. The positive power supply input of component U266 is also coupled by capacitor C261 to ground. The negative power supply input of component U266 is coupled to ground. The output of component U266 is coupled to pin 11 of component U264.

Impedance gain circuit 250 includes resistors R254-R259, capacitors C252-C255, and components U251-U253. Resistor R254 is coupled between the signal line PREAMP from the self test switch 225 (FIG. 7A) and a circuit node with the capacitor C252. The other side of the capacitor C252 is coupled to the negative input of component U251. Capacitor C253 and resistor R255 are coupled in parallel between the negative input of component U251 and the output of component of U251. The positive input of component U251 is coupled to ground. Resistor R256 is coupled between the output of component U251 and a circuit node with capacitor 254. The other side of capacitor 254 is coupled to the negative input of component U252.

Capacitor C255 and resistor R257 are coupled in parallel between the negative input of component U252 and the output of component U252. The positive input of component U252 is coupled to the voltage line BIAS. The output of component U252 is coupled to pins 2 and 4 of component 270. The output of component U252 is also coupled by resistor R258 to the negative input of component U253. The negative input of component U253 is coupled by resistor R259 to the output of component U253. The positive input of component U253 is coupled to the voltage line BIAS. The output of component U253 is coupled to pins 9 and 7 of impedance demodulator circuit 270.

Impedance demodulator circuit 270 has ten connected pins, including an IN1 pin 1, an S1A pin 2, a GND pin 3, an S2A pin 4, an IN2 pin 5, a D2 pin 6, an S2B pin 7, a VDD pin 8, an S1B pin 9, and a D1 pin 10. IN1 pin 1 is coupled to the signal line ZDM1. IN2 pin 5 is coupled to the signal line ZDM2. S1A pin 2 is coupled to S2A pin 4, which is also coupled to the output of component U252 of the impedance gain circuit 250. S1B pin 9 is coupled to S2B pin 7, which is also coupled to the output of component U253 of the impedance gain circuit 250. GND pin 3 is coupled to ground, while VDD pin 8 is coupled to the voltage line PAD+. VDD pin 8 is also coupled by capacitor C270 to ground D1 pin 10 is coupled to the impedance reactive filter circuit 280. D2 pin 6 is coupled to the impedance resistive filter circuit 285.

Impedance reactive filter circuit 280 and impedance resistive filter circuit 285 are of similar construction, and thus only the construction of impedance reactive filter circuit 280 will be described herein. Impedance reactive filter circuit 280 includes resistors R280-R284, capacitors C280-C281 and a component U280. Resistor R280 is coupled between D1 pin 10 of impedance demodulator 270 and a circuit node between capacitor C280 and resistor R281. The other side of capacitor C280 is coupled to ground, while the other side of resistor R281 is coupled to a circuit node with capacitor C282 and resistor R282. The other side of capacitor C282 is coupled to the output of component U280, while the other side of resistor R2822 is coupled to the positive input of component U280.

The positive input of component U280 is coupled by capacitor C281 to ground. The negative input of component U280 is coupled by resistor R283 to the output of component U280. The negative input of component U280 is also coupled by resistor R284 to the voltage line BIAS. The output of component U280 is coupled to the signal line Z-REACTIVE. As noted above, the impedance resistive filter circuit 285 is of similar construction to the impedance reactive filter circuit 280 and will not be described further herein, other than to note that within the impedance resistive filter circuit 285 the resistor R285 is coupled to the D2 pin 6 of the impedance demodulator 270, and the output of component U285 is coupled to the signal line Z-RESISTIVE.

Impedance motion filter 290 includes resistors R290-R295, capacitors C290-C293 and components U290 and U291. Capacitor C290 is coupled between the signal line Z-RESISTIVE and the positive input of component U290. The positive input of component U290 is also coupled by resistor R290 to the voltage line BIAS. The negative input of component U290 is coupled by resistor R291 to the voltage line BIAS. Capacitor C291 and resistor R292 are coupled in parallel between the negative input of component U290 and the output of component U290. Capacitor C292 is coupled between the output of component U290 and the positive input of component U291. The positive input of component U291 is coupled by resistor R293 to the voltage line BIAS. The negative input of component U291 is coupled by resistor R294 to the voltage line BIAS. Capacitor C293 and resistor R295 are coupled in parallel between the negative input of component U291 and the output of component U291. The output of component U291 is coupled to the signal line Z-MOTION.

FIG. 7C is a schematic diagram of the ECG A-to-D converter circuit 37c of FIG. 6. As shown in FIG. 7C, the ECG A-to-D converter circuit 37c includes resistors R296-R299, components U296-U299, and a capacitor C296. Signal line AD-DIN is coupled to the input of component U297. Resistor R297 is coupled between the input of component U297 and ground. A signal line AD-CLK is coupled to the input of component U298. Resistor R298 is coupled between the input of component U298 and ground. A signal line AD-CS is coupled to the input of component U299. Resistor R299 is coupled between the input of component U299 and ground. The output of component U297 is coupled to pin 17 of component U296. The output of component U298 is coupled to pin 19 of component U296. The output of component U299 is coupled to pin 18 of component U296.

Component U296 has nineteen connected pins, including a CH0 pin 1, a CH1 pin 2, a CH2 pin 3, a CH3 pin 4, a CH4 pin 5, a CH5 pin 6, a CH6 pin 7, a CH7 pin 8, a COM pin 9, an SHDN pin 10, a VREF pin 11, a VCC pin 12, a GND pin 13, a GND pin 14, a DOUT pin 15, a DIN pin 17, an SC pin 18, an SLC pin 19, and a VCC pin 20. CH0 pin 1 is coupled to the signal line ECG. CH1 pin 2 is coupled to the signal line Z-RESISTIVE. CH2 pin 3 is coupled to the signal line Z-REACTIVE. CH3 pin 4 is coupled to the signal line Z-MOTION. CH4 pin 5 is coupled to the voltage line 3.3V-A. CH5 pin 6 is coupled to the signal line TEMPERATURE. CH7 pin 8 is coupled to the signal line VCAP-HV2. CH7 pin 8 is coupled by resistor 296 to ground. COM pin 9 is coupled to GND pin 13 and to GND pin 14, each of which are coupled to ground. SHDN pin 10 is coupled to REF pin 11, which is coupled to the voltage line PAD+. VCC pin 12 is coupled to VCC pin 20, which is coupled to the voltage line PAD+. VCC pin 12 and VCC pin 20 are also coupled by capacitor C296 to ground. The OUT pin 15 is coupled to the signal line AD-DOUT. DIN pin 17 is coupled to the output of component U297. CS pin 18 is coupled to the output of component U299. CLK pin 19 is coupled to the output of component U298.

As described above, the preamplifier ECG and impedance drive and measurement circuit 37 is referenced to the same common ground as the output circuit 14 and charging circuit 18. In one embodiment, it will be understood that it is the preamplifier power supply that is referenced to the common ground, as opposed to the preamplifier input. In other words, the preamplifier power supply is not isolated from the output circuit 14. Thus, the power supplies are not electrically isolated from one another.

It will be appreciated that the defibrillator 8 described above with reference to FIGS. 1-7C provides a number of advantages over prior art defibrillators. The utilization of a common ground for the defibrillator results in a simpler circuit design than was required in prior art defibrillators which utilized isolation circuits for the high and low voltage circuitry. The utilization of the common ground also allows one or both of the SCRs in the two lower legs of the H-bridge to be driven with DC gate drive signals, thus reducing the complexity of the drive circuits. In addition, the placement of the IGBT in the northwest leg of the H-bridge is an improved design over prior art defibrillators which placed the IGBT in the southeast leg. These aspects of the design result in a defibrillator that is simpler, less expensive, and operates more effectively than prior art defibrillators.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A circuit for use in an external defibrillator, comprising:
at least one energy storage device having a first electrode and a second electrode;
a charging circuit coupled to the energy storage device, wherein the charging circuit is configured to charge the energy storage device;
an output circuit coupled to the energy storage device, the output circuit having a first output lead and a second output lead, and also having a plurality of output switches, wherein the output circuit is configured to switch the plurality of output switches to selectively electrically couple the first and second electrodes of the energy storage device to the first and second output leads; and
a control circuit coupled to the charging circuit and the output circuit, wherein the control circuit is configured to control the output circuit to couple the first and second electrodes of the energy storage device to the first and second output leads of the output circuit; and
a preamplifier circuit;
wherein the output circuit and preamplifier circuit are referenced to a common ground.

2. The circuit of claim 1, wherein the output circuit comprises an H-bridge output circuit and the plurality of output switches comprise:
(a) a first switch in the first leg of the H-bridge output circuit coupled between a first lead of the at least one energy storage device and the first electrode;
(b) a second switch in the second leg of the H-bridge output circuit coupled between a second lead of the at least one energy storage device and the second electrode;
(c) a third switch in the third leg of the H-bridge output circuit coupled between the first lead of the at least one energy storage device and the second electrode; and
(d) a fourth switch in the fourth leg of the H-bridge output circuit coupled between the second lead of the at least one energy storage device and the first electrode.

3. The circuit of claim 2, wherein the first switch comprises a single IGBT switch, and the first leg is an upper leg of the H-bridge output circuit.

4. The circuit of claim 3, wherein the second, third, and fourth switches each comprise an SCR switch.

5. The circuit of claim 2, wherein the output switches are driven so that they are capable of conducting a least approximately 200 amperes of current.

6. The circuit of claim 2, wherein the output circuit comprises at least one SCR switch and the control circuit includes a gate drive circuit for driving the gate of the at least one SCR switch with a gate drive signal.

7. The circuit of claim 6, wherein the gate drive signal comprises a direct drive current that is applied to the gate of the SCR switch.

8. The circuit of claim 7, wherein the continuous drive current is a DC current.

9. The circuit of claim 7, wherein the second and fourth legs of the H-bridge are the lower legs of the H-bridge, and the SCR is contained in one of the lower legs of the H-bridge and the other lower leg of the H-bridge also contains an SCR switch which is also driven by a continuous drive current.

10. The circuit of claim 2, wherein the four legs of the H-bridge output circuit each comprise a single output switch, and all four of the H-bridge output switches are contained within a single package.

11. The circuit of claim 1, wherein the charging circuit is referenced to the common ground.

12. The circuit of claim 1, wherein the control circuit is referenced to the common ground.

13. The circuit of claim 1, wherein the energy storage device is referenced to the common ground.

14. The circuit of claim 1, further comprising an impedance measurement circuit that is referenced to the common ground.

15. The circuit of claim 1, further comprising a battery that is referenced to the common ground.

16. In an external defibrillator with an H-bridge output circuit for applying a multiphasic defibrillation pulse to a patient through first and second electrodes when said first and second electrodes are coupled to a patient, said external defibrillator including an energy storage device having first and second leads, the H-bridge output circuit comprising:
(a) a first leg coupled between the first lead of the energy storage device and the first electrode, the first leg being an upper leg of the H-bridge output circuit;
(b) a second leg coupled between the second lead of the energy storage device and the second electrode, the second leg being a lower leg of the H-bridge output circuit;
(c) a third leg coupled between the first lead of the energy storage device and the second electrode, the third leg being an upper leg of the H-bridge output circuit;
(d) a fourth leg coupled between the second lead of the energy storage device and the first electrode, the fourth leg being a lower leg of the H-bridge output circuit; and
(e) wherein the first upper leg comprises an IGBT switch.

17. The output circuit of claim 16, wherein the second, third, and fourth legs each comprise an SCR switch.

18. The output circuit of claim 16, wherein the external defibrillator further comprises a preamplifier circuit, the preamplifier circuit being referenced to a common ground with the output circuit and the energy storage device.

19. The output circuit of claim 18, wherein the external defibrillator further comprises a charging circuit, the charging circuit being referenced to the common ground with the output circuit.

20. The output circuit of claim 16, wherein at least one of the second, third, or fourth legs comprises an SCR switch.

21. The output circuit of claim 20, wherein the SCR is driven by a gate drive signal, the gate drive signal comprising a direct drive current that is applied to the gate of the SCR switch.

22. The output circuit of claim 21, wherein the SCR switch is contained in one of the lower legs of the H-bridge, and the other lower leg of the H-bridge also contains an SCR switch which is also driven by a gate drive signal that comprises a direct drive current that is applied to the gate of the SCR switch.

23. In an external defibrillator with an H-bridge output circuit for applying a multiphasic defibrillation pulse to a patient through first and second electrodes when said first and second electrodes are coupled to a patient, said external defibrillator including an energy storage device having first and second leads, the H-bridge output circuit comprising:
(a) a first leg coupled between the first lead of the energy storage device and the first electrode, the first leg being an upper leg of the H-bridge output circuit;
(b) a second leg coupled between the second lead of the energy storage device and the second electrode, the second leg being a lower leg of the H-bridge output circuit;
(c) a third leg coupled between the first lead of the energy storage device and the second electrode, the third leg being an upper leg of the H-bridge output circuit;
(d) a fourth leg coupled between the second lead of the energy storage device and the first electrode, the fourth leg being a lower leg of the H-bridge output circuit; and
(e) wherein one of the first, second, third or fourth legs comprises an SCR switch, the SCR switch being driven by a gate drive signal, the gate drive signal comprising a direct drive current that is applied to the gate of the SCR switch.

24. The output circuit of claim 23, wherein the SCR switch is contained in one of the lower legs of the H-bridge, and one of the upper legs of the H-bridge comprises an IGBT switch.

25. A circuit for use in an external defibrillator, comprising:
at least one energy storage device having a first electrode and a second electrode;
a charging circuit coupled to the energy storage device, wherein the charging circuit is configured to charge the energy storage device;
an output circuit coupled to the energy storage device, the output circuit having a first output lead and a second output lead, and also having a plurality of output switches, wherein the output circuit is configured to switch the plurality of output switches to selectively electrically couple the first and second electrodes of the energy storage device to the first and second output leads; and
a control circuit coupled to the charging circuit and the output circuit, wherein the control circuit is configured to control the output circuit to couple the first and second electrodes of the energy storage device to the first and second output leads of the output circuit; and
a preamplifier circuit;
wherein the output circuit and preamplifier circuit are not electrically isolated from one another.
